(19) United States
(12) Patent Application Publication
(10) Pub. No.: US 2024/0024019 A1
(43) Pub. Date: Jan. 25, 2024

(54) COOLING SYSTEM FOR DISSECTION BLADE

(71) Applicant: Covidien LP, Mansfield, MA (US)

(72) Inventors: Richard L. Croft, Mead, CO (US); Matthew S. Cowley, Frederick, CO (US)

(21) Appl. No.: 18/268,376

(22) PCT Filed: Nov. 23, 2021
(86) PCT No.: PCT/US2021/060436

§ 371 Date: Jun. 20, 2023

Related U.S. Application Data
(60) Provisional application No. 63/128,212, filed on Dec. 21, 2020.

(51) Int. Cl. A61B 18/14

(52) U.S. Cl. CPC A61B 18/1442; A61B 2018/00017

(57) ABSTRACT

An end effector assembly includes first and second jaw members each having a tissue contacting surface and movable relative to one another between a spaced apart position and an approximated position for grasping tissue therebetween. An electromagnetic induction coil is fixedly disposed within the first jaw member. A thermal cutting element is disposed within the electromagnetic induction coil and is configured to protrude from the first jaw member and through the tissue contacting surface thereof. The thermal cutting element is formed from an electromagnetic material capable of being inductively heated. The electromagnetic induction coil is adapted to connect to a source of energy to produce an electromagnetic field within the electromagnetic induction coil to inductively heat the thermal cutting element. A cooling system is disposed within the first jaw member proximate the thermal cutting element and is configured to absorb heat from the thermal cutting element or actively cool the thermal cutting element after activation thereof.

### FIELD

The present disclosure relates to electrosurgical instruments and, more particularly, to electrosurgical instruments including thermal cutting elements to facilitate tissue treatment, e.g., sealing and cutting of tissue.

### BACKGROUND

A surgical forceps is a pliers like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife that is advanced between the jaw members to cut the treated tissue. As an alternative to a mechanical knife, energy-based tissue cutting may be employed to cut the treated tissue using energy, e.g., thermal, electrosurgical, ultrasonic, light, or other suitable energy.

### SUMMARY

As used herein, the term “distal” refers to the portion that is being described which is farther from an operator (whether a human surgeon or a surgical robotic), while the term “proximal” refers to the portion that is being described which is closer to the operator. Terms including “generally,” “about,” “substantially,” and the like, as utilized herein, are meant to encompass variations up to and including plus or minus 10 percent to take into account, for example, material, measurement, manufacturing, environmental, use, and/or other tolerances and variations. Further, to the extent consistent, any or all of the aspects detailed herein may be used in conjunction with any or all of the other aspects detailed herein.

Provided in accordance with aspects of the present disclosure is an end effector assembly having first and second jaw members each including a tissue contacting surface. One or both of the first or second jaw members is movable relative to the other between a spaced apart position and an approximated position for grasping tissue between the tissue contacting surfaces. An electromagnetic induction coil is fixedly disposed within the first jaw member. A thermal cutting element is disposed at least partially within the electromagnetic induction coil and is configured to protrude from the first jaw member and through or flush with the tissue contacting surface thereof. The thermal cutting element is formed at least partially from an electromagnetic material capable of being inductively heated. The electromagnetic induction coil is adapted to connect to a source of energy to produce an electromagnetic field within the electromagnetic induction coil to thereby inductively heat the thermal cutting element. A cooling system is disposed within the first jaw member proximate the thermal cutting element and is configured to absorb heat from the thermal cutting element or actively cool the thermal cutting element during or after activation thereof.

In aspects according to the present disclosure, the tissue contacting surfaces are formed from an electrically-conductive material and are adapted to connect to a source of energy for electrosurgically treating tissue grasped between the tissue contacting surfaces.

In aspects according to the present disclosure, the cooling system includes a tube configured to convey cooled air from an external source to the first jaw member, the tube extending parallel relative to the thermal cutting element along a substantial length thereof and configured to absorb heat from the thermal cutting element or actively cool the thermal cutting element during or after activation thereof. In other aspects according to the present disclosure, the tube of the cooling system includes a first conduit for conveying cooled air to the first jaw member and a second conduit for returning air to a collection tank.

In aspects according to the present disclosure, the second conduit includes one or more orifices disposed therealong configured to remove smoke from the surgical site under suction. In other aspects according to the present disclosure, the one or more orifices includes a venturi-shaped opening for suctioning air from the surgical site. In still other aspects according to the present disclosure, a pressurized air differential is used to evacuate external smoke through one or more orifices.

In aspects according to the present disclosure, the first conduit and second conduit are connected at a distal end thereof and an expansion nozzle is disposed between the first and second conduits. The expansion nozzle is configured to reduce the air pressure and increase the air velocity of the air flowing from the first conduit to the second conduit allowing the second conduit to absorb additional heat from the thermal cutting element or additionally actively cool the thermal cutting element (e.g., Joule-Thompson effect).

In aspects according to the present disclosure, the first conduit conveys compressed air to the first jaw member for absorption by the thermal cutting element.

In aspects according to the present disclosure, the thermal cutting element is formed at least partially from a ferromagnetic material.

Provided in accordance with other aspects of the present disclosure is an end effector assembly having first and second jaw members each including a tissue contacting surface. One or both of the first or second jaw members is movable relative to the other between a spaced apart position and an approximated position for grasping tissue between the tissue contacting surfaces. An electromagnetic induction coil is fixedly disposed within the first jaw member. A thermal cutting element is disposed at least partially within the electromagnetic induction coil and is configured to protrude from the first jaw member and through or flush with the tissue contacting surface thereof. The thermal cutting element is formed at least partially from an electromagnetic material capable of being inductively heated. The electromagnetic induction coil is adapted to connect to a source of energy to produce an electromagnetic field within the electromagnetic induction coil to thereby inductively heat the thermal cutting element.

A cooling system is disposed within the first jaw member proximate the thermal cutting element and has a tube including: a first conduit configured to convey compressed air from an external source therealong to absorb a first amount of heat from the thermal cutting element or actively cool the thermal cutting element a first amount during or after activation thereof; a second conduit configured to return the compressed air to a collection tank; and an expansion nozzle disposed between the first conduit and the second conduit that is configured to reduce the air pressure and increase the air velocity of the compressed air flowing from the first conduit to the second conduit allowing the second conduit to absorb a second amount of heat from the thermal cutting element or actively cool the thermal cutting element a second amount during or after activation thereof.

In aspects according to the present disclosure, the tissue contacting surfaces are formed from an electrically-conductive material and are adapted to connect to a source of energy for electrosurgically treating tissue grasped between the tissue contacting surfaces.

In aspects according to the present disclosure, the second conduit includes one or more orifices disposed therealong configured to remove smoke from the surgical site under suction. In other aspects according to the present disclosure, pressurized air is used with the venturi effect to evacuate external smoke through one or more orifices.

In aspects according to the present disclosure, the thermal cutting element is formed at least partially from a ferromagnetic material.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1A is a perspective view of a shaft-based electrosurgical forceps provided in accordance with the present disclosure wherein a shaft of the forceps is disposed in a non-articulated position and wherein jaw members of an end effector assembly of the forceps are disposed in a spaced apart position;

FIG. 1B is a perspective view of the forceps of FIG. 1A, wherein the shaft of the forceps is disposed in an articulated position and wherein the jaw members of the forceps are disposed in an approximated position;

FIG. 2 is a perspective view of a hemostat-style electrosurgical forceps provided in accordance with the present disclosure;

FIG. 3 is a schematic illustration of a robotic surgical system provided in accordance with the present disclosure;

FIG. 4 is a longitudinal, cross-sectional view of the end effector assembly of the forceps of FIG. 1A, configured for use with the hemostat-style electrosurgical forceps of FIG. 2, configured for use the robotic surgical system of FIG. 3, or configured for use with any other suitable surgical instrument or system, wherein a thermal cutting element thereof is disposed in a retracted position;

FIG. 5 is a longitudinal, cross-sectional view of the end effector assembly of FIG. 4, wherein the thermal cutting element is disposed in an extended position; and

FIG. 6 is a longitudinal, cross-sectional view of another embodiment of the end effector assembly including a static cutting element and an internal cooling system associated therewith.

### DETAILED DESCRIPTION

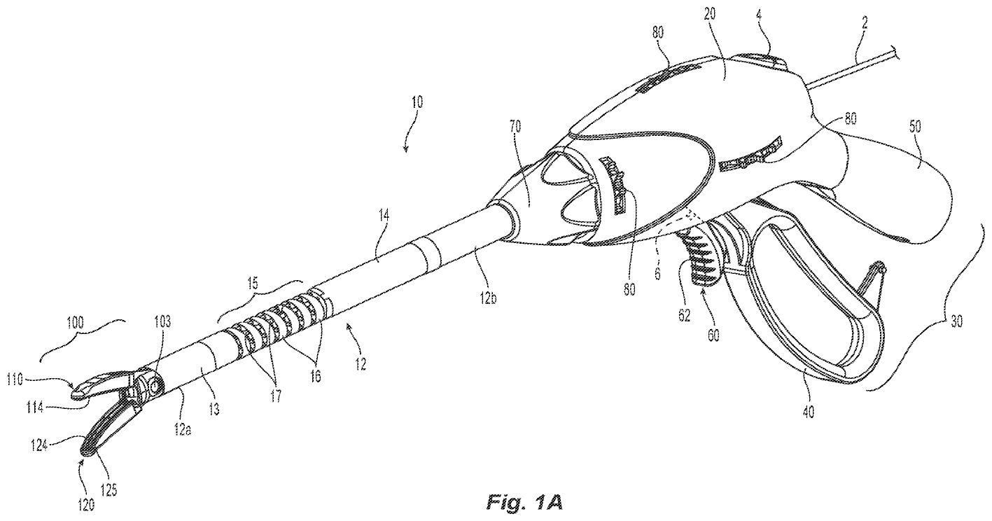

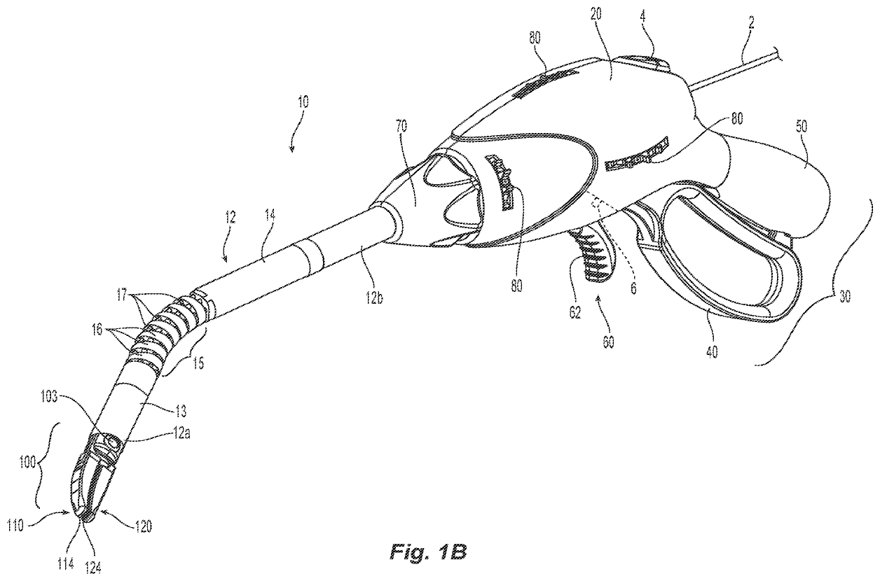

Turning to FIGS. 1A and 1B, a shaft based electrosurgical forceps provided in accordance with the present disclosure is shown generally identified by reference numeral 10. For the purposes herein, forceps 10 is generally described. Aspects and features of surgical forceps 10 not germane to the understanding of the present disclosure are omitted to avoid obscuring the aspects and features of the present disclosure in unnecessary detail.

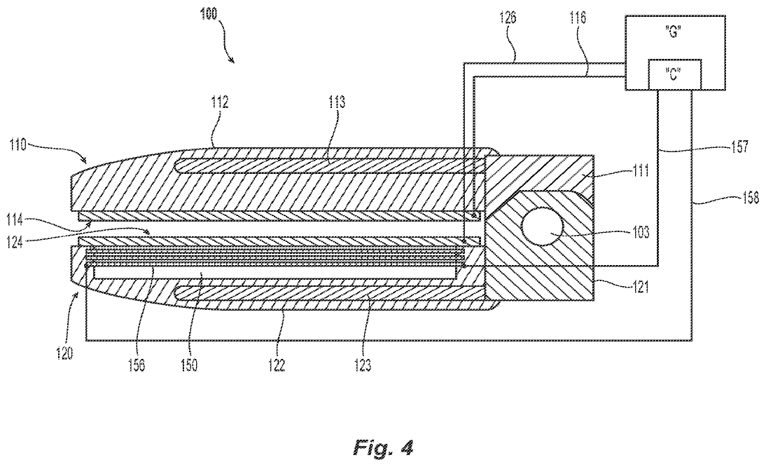

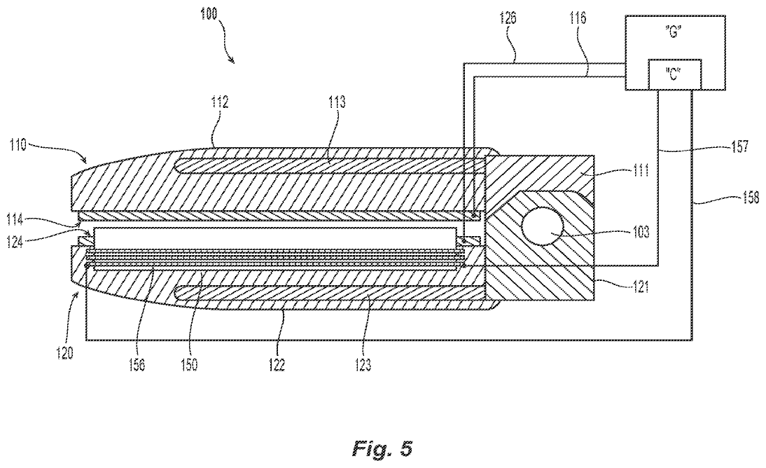

Forceps 10 includes a housing 20, a handle assembly 30, a trigger assembly 60, a rotating assembly 70, a plurality of articulation actuators 80, one or more activation switches 4, 6, and an end effector assembly 100. Forceps 10 further includes a shaft 12 having a distal end portion 12a configured to mechanically engage end effector assembly 100 and a proximal end portion 12b that mechanically engages housing 20. Forceps 10 also includes cable 2 that connects forceps 10 to an energy source, e.g., a generator “G” (FIGS. 4 and 5) or other suitable energy source, although forceps 10 may alternatively be configured as a battery-powered device including an on-board generator. Cable 2 includes a plurality of wires (not shown) extending therethrough that have sufficient length to extend into housing 20 and through shaft 12 in order to provide energy to one or both tissue contacting surfaces 114, 124 ofjaw members 110, 120, respectively, of end effector assembly 100 to enable electrosurgical tissue treatment, e.g., sealing, cauterizing, coagulating/desiccating, etc., of tissue grasped between tissue contacting surfaces 114, 124. Similar or different wires (not shown) of cable 2 extend into housing 20 and through shaft 12 in order to enable energization of thermal cutting element 150 (FIGS. 4 and 5) of end effector assembly 100 for thermally treating, e.g., cutting, spot cauterizing or coagulating, etc., tissue in contact with thermal cutting element 150 (FIGS. 4 and 5). Similar or different wires (not shown) also electrically couple the one or more activation switches 4, 6 of forceps 10 to tissue contacting surfaces 114, 124, thermal cutting element 150 (FIGS. 4 and 5), and/or the source of energy, e.g., generator “G” (FIGS. 4 and 5), to enable selective initiation of electrosurgical tissue treatment and/or thermal tissue treatment.

In some configurations, a single activated position activation switch 4 may be provided to automatically initiate electrosurgical tissue treatment (e.g., tissue sealing) and thermal tissue treatment (e.g., tissue cutting) upon activation, e.g., to first initiate sealing and, once sealing is complete, initiate cutting. Suitable feedback, sensors, or other suitable mechanism(s) to determine seal completion and to initiate cutting when seal completion is determined may be utilized. Alternatively, suitable feedback, sensors, or other suitable mechanism(s) may be utilized to determine instrument state (e.g., jaws open, jaws closed, thermal cutting element retracted, thermal cutting element deployed, etc.), tissue presence, a position/type/thickness of tissue, whether the end effector assembly is stationary or moving, whether sealing has been completed, etc., and, based thereon, automatically initiate or deactivate sealing or cutting. Further still, activation switch 4 may be a multi-activated position switch wherein each position corresponds to a different mode of operation, e.g., a first position for sealing and a second activation position for cutting. In yet other configurations, multiple different activation switches 4, 6 may be provided, e.g., one for initiating sealing and another for initiating cutting.

Shaft 12 of forceps 10 defines a distal segment 13 positioned towards distal end portion 12a thereof, a proximal segment 14 positioned towards proximal end portion 12b thereof, and an articulating section 15 disposed between the distal and proximal segments 13, 14, respectively. Articulating section 15 includes at least one articulating link 16 having a plurality of articulation cables 17 extending therethrough. Each cable 17 is operably engaged at its distal end to distal segment 13 and at its proximal end to one of the articulation actuators so as to enable articulation of distal segment 13 and, thus, end effector assembly 100, relative to proximal segment 14 upon actuation of one or more of articulation actuators 80. In some aspects, articulating section 15 and articulation actuators 80 are omitted such that shaft 12 of forceps 10 does not articulate. In either configuration, rotating assembly 70 operably couples shaft 12 to housing 20 so as to enable selective rotation of shaft 12 and, thus, end effector assembly 100, relative to housing 20.

Handle assembly 30 of forceps 10 includes a fixed handle 50 and a movable handle Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced apart position (FIG. 1A) and an approximated position (FIG. 1B) to grasp tissue between tissue contacting surfaces 114, 124 of jaw members 110, 120, respectively. As shown in FIG. 1A, movable handle 40 is initially spaced-apart from fixed handle 50 and, correspondingly, jaw members 110, 120 of end effector assembly 100 are disposed in the spaced-apart position. Movable handle 40 is depressible from this initial position to a depressed position corresponding to the approximated position of jaw members 110, 120 (FIG. 1B).

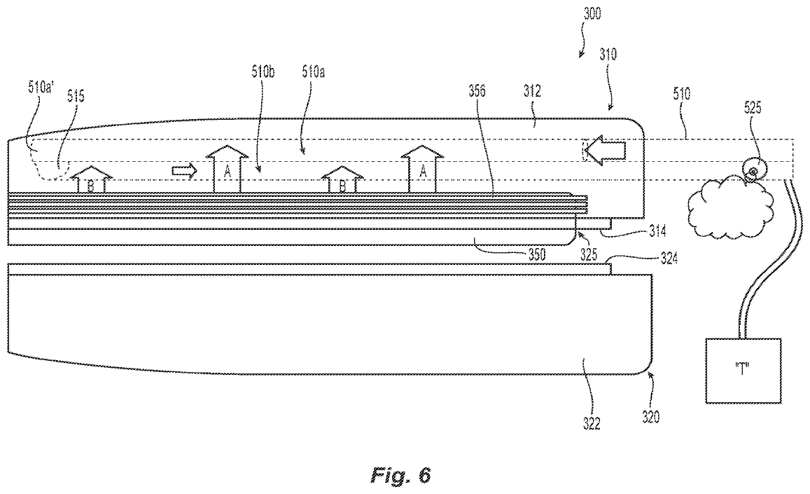

Trigger assembly 60 includes a trigger 62 coupled to housing 20 and movable relative thereto between an un-actuated position and an actuated position. Trigger 62 is operably coupled to a deployment mechanism, various configurations of which are detailed below, so as to enable selective deployment of thermal cutting element 150 (FIGS. 4 and 5) from a retracted position (FIG. 4), wherein thermal cutting element 150 (FIGS. 4 and 5) is flushed with or recessed within jaw member 120 (see FIG. 4), to an extended position (FIG. 5), wherein thermal cutting element 150 (FIGS. 4 and 5) protrudes upwardly from tissue contacting surface 124 of jaw member 120 (see FIG. 5), e.g., through a channel 125 defined within tissue contacting surface 124, to enable thermal cutting of tissue. As explained below in FIG. 6, another embodiment of the end effector 300 includes a static thermal cutting element 350.

As an alternative to a pivoting trigger 62, a slide trigger, push-button, toggle switch, or other suitable actuator may be provided. Further, in aspects where multiple activation switches 4, 6 are provided, the switch, e.g., switch 6, associated with initiating thermal cutting may be positioned in the actuation path of trigger 62 such that, initially upon movement of trigger 62 from the un-actuated position, upon completion of movement of trigger 62 from the un-actuated position to the actuated position, or at any suitable point along the actuation path of trigger 62 from the un-actuated position to the actuated position, switch 6 is activated to initiate thermal cutting. Switch 6 may be similarly be deactivated upon return of trigger 62 to the un-actuated position.

End effector assembly 100, as noted above, includes first and second jaw members 110, 120 pivotably coupled to one another about pivot 103 for moving one or both of jaw members 110, 120 relative to the other between the spaced-apart and approximated positions. Each jaw member 110, 120 includes a tissue contacting surfaces 114, 124, respectively, thereon, and one or both of the jaw members 110, 120, e.g., jaw member 120, includes a thermal cutting element 150 (FIGS. 4 and 5) selectively deployable therefrom to enable thermal cutting of tissue. Jaw members 110, 120 may define curved configurations wherein each jaw member is similarly curved laterally off of a longitudinal axis of end effector assembly 100. However, other suitable curved configurations including curvature towards one of the jaw members 110, 120 (and thus away from the other), multiple curves with the same plane, and/or multiple curves within different planes are also contemplated. Alternatively, jaw members 110, 120 may define straight or angled configurations. End effector assembly 100 is described in greater detail below with reference to FIGS. 4 and 5.

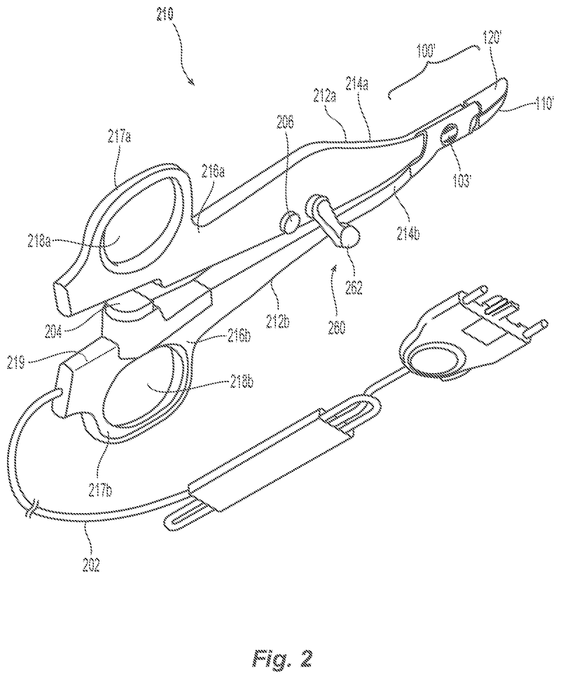

Referring to FIG. 2, a hemostat-style electrosurgical forceps provided in accordance with the present disclosure is shown generally identified by reference numeral 210. For the purposes herein, surgical forceps 210 is generally described. Aspects and features of open surgical forceps 210 not germane to the understanding of the present disclosure are omitted to avoid obscuring the aspects and features of the present disclosure in unnecessary detail.

Forceps 210 includes two elongated shafts 212a, 212b, each having a proximal end portion 216a, 216b, and a distal end portion 214a, 214b, respectively. Forceps 210 is configured for use with an end effector assembly 100′ similar to and including any of the features of end effector assembly 100 (FIGS. 1A, 1B, 4, and 5). More specifically, end effector assembly 100′ includes first and second jaw members 110′, 120′ attached to respective distal end portions 214a, 214b of shafts 212a, 212b and movable about a pivot 103′ to grasp, treat, e.g., seal, and/or thermally cut tissue. Each shaft 212a, 212b includes a handle 217a, 217b disposed at the proximal end portion 216a, 216b thereof. Each handle 217a, 217b defines a finger hole 218a, 218b therethrough for receiving a finger of the user. As can be appreciated, finger holes 218a, 218b facilitate movement of the shafts 212a, 212b relative to one another to, in turn, pivot jaw members 110′, 120′ from the spaced-apart position, wherein jaw members 110′, 120′ are disposed in spaced relation relative to one another, to the approximated position, wherein jaw members 110′, 120′ cooperate to grasp tissue therebetween.

One of the shafts 212a, 212b of forceps 210, e.g., shaft 212b, includes a proximal shaft connector 219 configured to connect forceps 210 to a source of energy, e.g., generator “G” (FIGS. 4 and 5) or other suitable energy source. Proximal shaft connector 219 secures a cable 202 to forceps 210 such that the user may selectively supply energy to jaw members 110′, 120′ for treating tissue and for energy-based tissue cutting. More specifically, one or more activation switches 204, 206 are provided for supplying energy to jaw members 110′, 120′ to electrosurgically treat, e.g., seal, tissue and/or to a thermal cutting element (not shown, see thermal cutting element 150 (FIGS. 4 and 5)) of end effector assembly 100′ to thermally treat, e.g., cut, tissue. Activation switch 204 may be positioned to be activated upon sufficient approximation of shaft members 212a, 212b to initiate electrosurgical treatment, while activation switch 206 may be activated during initiation, completion, or other portion of actuation of trigger 262 to initiate thermal treatment. Other suitable configurations of one or more activation switches 204, 206 such as those detailed above with respect to activation switches 4, 6 (FIGS. 1A and 1B) are also contemplated.

Forceps 210 further includes a trigger assembly 260 including a trigger 262 coupled to one of the shafts, e.g., shaft 212a, and movable relative thereto between an un-actuated position and an actuated position. Trigger 262 is operably coupled to a deployment mechanism, various configurations of which are detailed below, so as to enable selective deployment of a thermal cutting element (not shown, see thermal cutting element 150 (FIGS. 4 and 5)) between the tissue contacting surfaces of jaw members 110′, 120′ to thermally cut tissue grasped between jaw members 110,′ 120′. Similarly as above, as an alternative to a pivoting trigger 262, a slide trigger, push-button, toggle switch, or other suitable actuator may be provided.

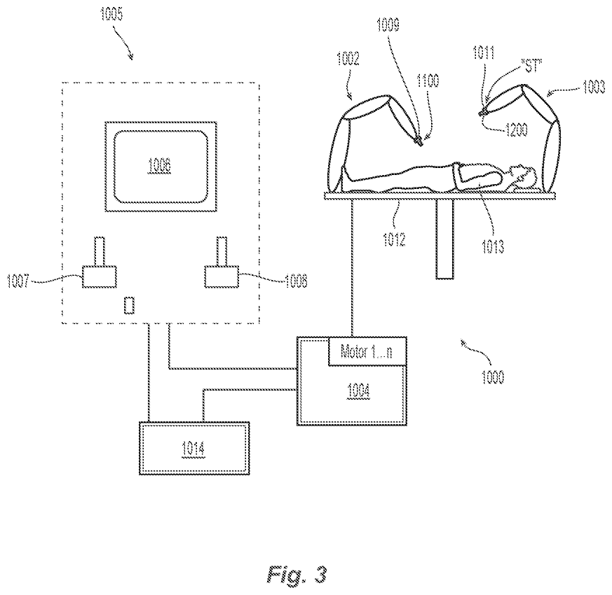

Referring generally to FIG. 3, a robotic surgical system provided in accordance with the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include one or more sections, which are connected through joints, and an attaching device 1009, 1011, to which may be attached, for example, an end effector assembly 1100, 1200, respectively. End effector assembly 1100 may be similar to and include any of the features of end effector assembly 100 (FIGS. 1A, 1B, 4, and 5), although other suitable end effector assemblies for coupling to attaching device 1009 are also contemplated. End effector assembly 1200 may be any suitable end effector assembly, e.g., an endoscopic camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

Referring to FIGS. 4 and 5, end effector assembly 100 of shaft-based electrosurgical forceps 10 (FIGS. 1A and 1B) is described in more detail, although end effector assembly 100 may equally be applicable for use with hemostat-style electrosurgical forceps 210 (FIG. 2), robotic surgical system 1000 (FIG. 3), or any other suitable surgical instrument or system.

End effector assembly 100, as noted above, includes first and second jaw members 110, 120. Each jaw member 110, 120 includes a proximal flag portion 111, 121, an outer insulative jaw housing 112, 122, a structural body 113, 123, and a tissue contacting surface 114, 124, respectively. Proximal flag portions 111, 121 are pivotably coupled to one another about a pivot 103 to enable movement of one or both of jaw members 110, 120 relative to the other between the spaced-apart and approximated positions. Any suitable mechanism for pivoting jaw members 110, 120 relative to one another about pivot 103 may be utilized. Structural bodies 113, 123 may be formed with proximal flag portions 111, 121 or separate therefrom and, in either configuration, extend distally from proximal flag portions 111, 121 to support jaw housings 112, 122 and tissue contacting surfaces 114, 124, respectively, thereon.

Tissue contacting surfaces 114, 124 are connected to generator “G,” e.g., via leads 116, 126, and are formed from electrically conductive material(s) to enable electrosurgical treatment of tissue grasped therebetween. For example, generator “G” may be configured to energize tissue contacting surfaces 114, 124 with Radio Frequency (RF) electrosurgical energy at different potentials to establish a potential gradient for conducting electrosurgical energy therebetween and through grasped tissue to electrosurgically treat, e.g., seal, tissue. Tissue contacting surfaces 114, 124 may alternatively be configured to supply or conduct any other suitable electrosurgical energy, e.g., microwave, light, ultrasonic, etc., to or through tissue grasped therebetween for electrosurgical tissue treatment. Tissue contacting surfaces 114, 124 may be defined on plates secured to jaw housings 112, 122, respectively, may be deposited onto jaw housings 112, 122, e.g., via sputtering or other suitable deposition technique, or may define any other suitable configuration. One or more stops (not shown) configured to inhibit shorting between tissue contacting surfaces 114, 124 may be disposed on either or both tissue contacting surfaces 114, 124.

Continuing with reference to FIGS. 4 and 5, one or both of the jaw members 110, 120, e.g., jaw member 120, includes a thermal cutting element 150 disposed therein and selectively deployable from a retracted position (FIG. 4), wherein thermal cutting element 150 is flush with or recessed within jaw member 120 (and does not protrude from tissue contacting surface 124), to an extended position (FIG. 5), wherein thermal cutting element 150 protrudes upwardly from tissue contacting surface 124 of jaw member 120, e.g., through channel 125 (FIGS. 1A and 1B) defined within tissue contacting surface 124 towards tissue contacting surface 114 of jaw member 110 (FIGS. 1A and 1B). In some configurations, tissue contacting surface 114 defines a channel (not shown, similar to channel 125 (FIGS. 1A and 1B)) for receipt of at least a portion of thermal cutting element 150 therein in the extended position of thermal cutting element 150 and the approximated position of jaw members 110, 120.

Thermal cutting element 150 defines an elongated configuration extending, in aspects, at least 85%, in other aspects at least 90%, and in still other aspects, at least 95% of the length of tissue contacting surface 124, although other configurations are also contemplated. In this manner, thermal cutting element 150 is capable of fully dividing sealed tissue grasped between tissue contacting surfaces 114, 124 regardless of the position of the sealed tissue or the length of sealed tissue. It is noted that, even though thermal cutting element 150 may not extend the entire length of tissue contacting surface 124, some tissue cutting may be enabled beyond the length of thermal cutting element 150, thus enabling tissue cutting the full length of tissue contacting surface 124. In other aspects, thermal cutting element 150 extends a smaller portion of the length of tissue contacting surface, the entire length of tissue contacting surface 124, or beyond the length of tissue contacting surface 124, e.g., to protrude distally therefrom to define a thermal probe to facilitate blunt dissection, spot cauterization or coagulation, enterotomies, etc. In any of the above-noted aspects, multiple thermal cutting elements 150 may be arranged lengthwise along jaw member 120 such that the thermal cutting elements 150 collectively define the desired length. In such aspects, the thermal cutting elements 150 may be independently deployable and/or activatable, or collectively deployable and/or activatable.

Thermal cutting element 150 may be formed from an electromagnetic material, e.g., a metal, and is configured to be inductively heated via a coil 156 disposed within outer jaw housing 122 of jaw member 120. In aspects, thermal cutting element 150 is formed from a ferromagnetic material.

Referring still to FIGS. 4 and 5, as noted above, a coil 156 is disposed within outer jaw housing 122 of jaw member 120. More specifically, coil 156 is an elongated electromagnetic induction coil 156 disposed within outer jaw housing 122 in fixed position therein (although, in some aspects, coil 156 may be movable with thermal cutting element 150 and relative to jaw member 120). Coil 156 is positioned to surround thermal cutting element 150 in a lengthwise direction and defines a height that extends at least a portion of a height of thermal cutting element 150, e.g., at least 40%, at least 50%, at least 60%, or any other suitable portion of the (or the entire) height of thermal cutting element 150. Coil 156 is configured to at least partially overlap with a height of thermal cutting element 150 in each of the retracted (FIG. 4) and extended (FIG. 5) positions. Coil 156 is further configured to electrically couple to generator “G” and, more specifically, a pair of electrical leads 157, 158 that are electrically isolated from one another are configured to connect to generator “G” and first and second end portions of coil 156 to enable energization of coil 156. When coil 156 is energized via energy from generator “G,” coil 156 produces an electromagnetic field therein, which serves to inductively heat thermal cutting element 150.

The inductance and resistance of coil 156 are functions of the permeability of thermal cutting element 150. With respect to a ferromagnetic material(s) forming thermal cutting element 150, for example, permeability varies with temperature. From room temperature, for example, the permeability of a ferromagnetic material increases as temperature increases until reaching the Curie temperature, at which point the permeability decreases sharply to a substantially paramagnetic state. Thus, automatic, Curie-point temperature control may be implemented wherein thermal cutting element 150 is heated to and maintained at its Curie temperature by this variation in inductance or resistance as a function of temperature. Alternatively or additionally, this variation in inductance or resistance as a function of temperature can be used for temperature control at other temperatures and/or for temperature measurement (and control based thereon). That is, changes in inductance or resistance can be detected as changes in voltage, current, and/or phase angle between coil voltage and current, e.g., via generator “G”, thus enabling temperature measurement. Feedback based voltage and/or current control (e.g., utilizing feedback as to voltage, current, and/or phase angle) can also be used to control heating and maintain thermal cutting element 150 at a target temperatures below its Curie point and/or to follow a heating temperature profile.

In aspects, control may be implemented by establishing an inductance-capacitance (LC) circuit from which oscillation frequency is derived. By providing a capacitor “C,” e.g., within generator “G” or otherwise positioned, and with the inductance being the inductance of coil 156 and thermal cutting element 150, temperature can be determined based on the fact that the frequency of oscillation of the LC circuit is a function of temperature. Thus, at room temperature of thermal cutting element 150, for example, the LC circuit oscillates at a relatively low frequency. As coil 156 inductively heats thermal cutting element 150, the oscillation frequency decreases until thermal cutting element 150 reaches its Curie point temperature, at which time the oscillation frequency jumps to a relatively high frequency. The oscillation frequency of the LC circuit thus changes based upon the temperature of the thermal cutting element 150 and, thus, enables temperature control based on monitoring oscillation frequency. This variation in oscillation frequency as a function of temperature can be used to implement temperature control or temperature measurement, similarly as detailed above with respect to inductance variation as a function of temperature.

In aspects, the heating of thermal cutting element 150 and the deployment of thermal cutting element 150 may be independent of one another; in other aspects, the heating of thermal cutting element 150 and the deployment of thermal cutting element 150 may be coupled to one another. For example: deployment of thermal cutting element 150 to the extended position may initiate heating of thermal cutting element 150 (at the beginning of deployment, after completion of deployment, or at any other position therebetween), e.g., as detailed above with respect to switch 6 and trigger 62 (FIGS. 1A and 1B); return of thermal cutting element 150 to the retracted position may deactivate heating of thermal cutting element 150 (at the beginning of return, after completion of return, or at any other position therebetween) e.g., likewise utilizing switch 6 and trigger 62 (FIGS. 1A and 1B); heating of thermal cutting element 150 may be inhibited until thermal cutting element 150 is deployed to the extended position; heating of thermal cutting element 150 may be inhibited when thermal cutting element 150 is not disposed in the extended position; deployment of thermal cutting element 150 to the extended position may be inhibited until thermal cutting element 150 is sufficiently heated (e.g., to its Curie temperature, other temperature set-point, a minimum temperature threshold, etc.); return of thermal cutting element 150 to the retracted position may be inhibited until thermal cutting element 150 is sufficiently cooled; etc.

Thermal cutting element 150, in the extended position (FIG. 5), and/or while moving to the extended position (FIG. 5), may be utilized to statically and/or dynamically thermally treat, e.g., cut, tissue grasped between jaw members 110, 120 (for example, after sealing tissue); may be utilized to statically and/or dynamically thermally treat tissue in a jaw members 110, 120 open condition, e.g., via tenting; and/or may be utilized in any other suitable matter to facilitate static and/or dynamic thermal tissue treatment.

Turning to FIG. 6, end effector assembly 300 of shaft-based electrosurgical forceps 10 (FIGS. 1A and 1B) is described in more detail, although end effector assembly 300 may equally be applicable for use with hemostat-style electrosurgical forceps 210 (FIG. 2), robotic surgical system 1000 (FIG. 3), or any other suitable surgical instrument or system.

End effector assembly 300, as noted above, includes similar element to the end effector assembly 200 described above with respect to FIGS. 4 and 5 and, as such, only those elements that differ are described in further detail herein. Any suitable mechanism for pivoting jaw members 310, 320 relative to one another about pivot, e.g., pivot 103, may be utilized. Tissue contacting surfaces 314, 324 are connected to generator “G” (FIGS. 4 and 5), e.g., via leads, e.g., leads 116, 126, and are formed from electrically conductive material(s) to enable electrosurgical treatment of tissue grasped therebetween. Generator “G” may be configured to energize tissue contacting surfaces 314, 324 with Radio Frequency (RF) electrosurgical energy at different potentials to establish a potential gradient for conducting electrosurgical energy therebetween and through grasped tissue to electrosurgically treat, e.g., seal, tissue. Tissue contacting surfaces 114, 124 may alternatively be configured to supply or conduct any other suitable electrosurgical energy, e.g., microwave, light, ultrasonic, etc., to or through tissue grasped therebetween for electrosurgical tissue treatment.

Tissue contacting surfaces 314, 324 are disposed in jaw housings 312, 322, respectively, or may be deposited onto jaw housings 312, 322, e.g., via sputtering or other suitable deposition technique, or may define any other suitable configuration. One or more stops (not shown) configured to inhibit shorting between tissue contacting surfaces 314, 324 may be disposed on either or both tissue contacting surfaces 314, 324.

One or both of the jaw members 310, 320, e.g., jaw member 310, includes a thermal cutting element 350 disposed therein which may extend from tissue contacting surface 314 or reside substantially flush therewith. Thermal cutting element 350 may be centrally disposed within tissue contacting surface 314 along the length thereof or off center depending upon a particular purpose. Thermal cutting element 350 extends from tissue contacting surface 314 of jaw member 310, e.g., through a channel 325 defined within tissue contacting surface 314 and into housing 312. Thermal cutting element 350 may be secured within the jaw housing 312 via overmolding, mechanical attachment or any other way known in the art.

Similar to thermal cutting element 150, thermal cutting element 350 defines an elongated configuration extending, in aspects, at least 85%, in other aspects at least 90%, and in still other aspects, at least 95% of the length of tissue contacting surface 314, although other configurations are also contemplated. In this manner, thermal cutting element 350 is capable of fully dividing sealed tissue grasped between tissue contacting surfaces 314, 324 regardless of the position of the sealed tissue or the length of sealed tissue. It is noted that, even though thermal cutting element 350 may not extend the entire length of tissue contacting surface 314, some tissue cutting may be enabled beyond the length of thermal cutting element 350, thus enabling tissue cutting the full length of tissue contacting surface 314. In other aspects, thermal cutting element 350 extends a smaller portion of the length of tissue contacting surface 314, the entire length of tissue contacting surface 314, or beyond the length of tissue contacting surface 314, e.g., to protrude distally therefrom to define a thermal probe to facilitate blunt dissection, spot cauterization or coagulation, enterotomies, etc. In any of the above-noted aspects, multiple thermal cutting elements 350 may be arranged lengthwise along jaw member 310 such that the thermal cutting elements 350 collectively define the desired length.

Thermal cutting element 350 may be formed from an electromagnetic material, e.g., a metal, and is configured to be inductively heated via a coil 356 disposed within outer jaw housing 312 of jaw member 310. In aspects, thermal cutting element 350 is formed from a ferromagnetic material.

Referring still to FIG. 6, a coil 356 is disposed within outer jaw housing 312 of jaw member 310. Similar to coil 156, coil 356 is an elongated electromagnetic induction coil 356 disposed within outer jaw housing 312 in fixed position therein. Coil 356 is positioned to surround thermal cutting element 350 in a lengthwise direction and defines a height that extends at least a portion of a height of thermal cutting element 150, e.g., at least 40%, at least 50%, at least 60%, or any other suitable portion of the (or the entire) height of thermal cutting element 350. Coil 356 is configured to at least partially overlap with a height of thermal cutting element 350. Coil 356 is further configured to electrically couple to generator “G” and, more specifically, a pair of electrical leads, e.g., similar to leads 157, 158 of FIGS. 4 and 5, that are electrically isolated from one another are configured to connect to generator “G” and first and second end portions of coil 356 to enable energization of coil 356. When coil 356 is energized via energy from generator “G,” coil 356 produces an electromagnetic field therein, which serves to inductively heat thermal cutting element 350.

As discussed above, the inductance and resistance of coil 356 are functions of the permeability of thermal cutting element 150. With respect to a ferromagnetic material(s) forming thermal cutting element 350, for example, permeability varies with temperature. Thus, automatic, Curie-point temperature control may be implemented wherein thermal cutting element 350 is heated to and maintained at its Curie temperature by this variation in inductance or resistance as a function of temperature. Feedback based voltage and/or current control (e.g., utilizing feedback as to voltage, current, and/or phase angle) can also be used to control heating and maintain thermal cutting element 350 at a target temperatures below its Curie point and/or to follow a heating temperature profile. As discussed above, control may be implemented by establishing an inductance-capacitance (LC) circuit from which oscillation frequency is derived. By providing a capacitor “C,” e.g., within generator “G” or otherwise positioned, and with the inductance being the inductance of coil 356 and thermal cutting element 350, temperature can be determined based on the fact that the frequency of oscillation of the LC circuit is a function of temperature. This variation in oscillation frequency as a function of temperature can be used to implement temperature control or temperature measurement, similarly as detailed above with respect to inductance variation as a function of temperature.

Thermal cutting element 150 may be utilized to statically thermally treat, e.g., cut, tissue grasped between jaw members 310, 320 (for example, after sealing tissue); may be utilized to statically thermally treat tissue in a jaw members 310, 320 open condition, e.g., via tenting; and/or may be utilized in any other suitable matter to facilitate static thermal tissue treatment.

During sealing and cutting tissue, there is a tendency for the thermal cutting element 350 to retain heat for prolonged periods of time reducing the effectiveness of subsequent seals. End effector assembly 300 includes an active heating system 500 disposed within one or both jaw members 310, 320. Heating system 500 includes a multi-conduit tube 510 extending through the shaft 12 and configured to operably engage the end effector assembly 300 (or otherwise externally couple to the end effector assembly 300). Tube 510 is disposed proximate thermal cutting element 350 and is configured to extend lengthwise at least along a major portion of the thermal cutting element 350. A first conduit 510a of tube 510 supplies a pressurized or compressed flow of air (or another gas) from a source (not shown) towards the thermal cutting element 350. Sources may include an external pump or tank or a small pump or canister internal to the instrument 10. The first conduit 510a is configured to absorb heat (See arrows “A”) from the thermal cutting element 350 or actively cool the thermal cutting element 350 as the compressed air passes through the first conduit 510a proximate the thermal cutting element 350.

A distal end 510a′ of the first conduit includes an expansion nozzle 515 defined therein disposed between the first conduit and a second air conduit 510b disposed in the tube 510. Expansion nozzle 515 is configured to reduce the pressure of the compressed air, increase the velocity of the compressed air flowing therethrough and into the second conduit 510b and cool the air returning to a smoke evacuation unit or collection container or canister “T”. The second conduit 510b provides a second opportunity for the absorption of heat (See arrows “B”) from the thermal heating element 350 or active cooling of the thermal cutting element 350 as the high velocity air returns passed the thermal heating element 350 for collection.

The second conduit 510b may include a series of small orifices 525 defined along a length thereof that are configured to suck in smoke and small surgical debris during the sealing and cutting process. The small orifices 525 may be configured as to produce a Venturi-like suction effect therethrough. The air and smoke continues through the second conduit 510b to the smoke evacuation unit or collection canister or tank (Not shown). By cooling the thermal cutting element 350 after each use, more efficient and faster seals may be achieved with reduced smoke, especially when sealing smaller vessels and avascular tissue.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. An end effector assembly, comprising:
first and second jaw members each including a tissue contacting surface, at least one of the first or second jaw members movable relative to the other between a spaced apart position and an approximated position for grasping tissue between the tissue contacting surfaces;
an electromagnetic induction coil fixedly disposed within the first jaw member;
a thermal cutting element disposed at least partially within the electromagnetic induction coil and configured to protrude from the first jaw member and through the tissue contacting surface thereof, the thermal cutting element formed at least partially from an electromagnetic material capable of being inductively heated, and wherein the electromagnetic induction coil is adapted to connect to a source of energy to produce an electromagnetic field within the electromagnetic induction coil to thereby inductively heat the thermal cutting element; and
a cooling system disposed within the first jaw member proximate the thermal cutting element, the cooling system configured to absorb heat from the thermal cutting element during or after activation thereof.

2. The end effector assembly according to claim 1, wherein the tissue contacting surfaces are formed from an electrically-conductive material and adapted to connect to a source of energy for electrosurgically treating tissue grasped between the tissue contacting surfaces.

3. The end effector assembly according to claim 1, wherein the cooling system includes a tube configured to convey cooled air from an external source to the first jaw member, the tube extending parallel relative to the thermal cutting element along a substantial length thereof and configured to absorb heat from the thermal cutting element during or after activation thereof.

4. The end effector assembly according to claim 3, wherein the tube of the cooling system includes a first conduit for conveying cooled air to the first jaw member and a second conduit for returning air to a collection tank.

5. The end effector assembly according to claim 4, wherein the second conduit includes at least one orifice disposed therealong configured to remove smoke from the surgical site under suction.

6. The end effector assembly according to claim 5, wherein the at least one orifice includes a venturi-shaped opening for suctioning air from the surgical site.

7. The end effector assembly according to claim 3, wherein the first conduit and second conduit are connected at a distal end thereof and an expansion nozzle is disposed between the first and second conduits, the expansion nozzle configured to reduce the air pressure and increase the air velocity of the air flowing from the first conduit to the second conduit allowing the second conduit to absorb additional heat from the thermal cutting element.

8. The end effector assembly according to claim 3, wherein the first conduit conveys compressed air to the first jaw member for absorption by the thermal cutting element.

9. The end effector assembly according to claim 1, wherein the thermal cutting element is formed at least partially from a ferromagnetic material.

10. An end effector assembly, comprising:
first and second jaw members each including a tissue contacting surface, at least one of the first or second jaw members movable relative to the other between a spaced apart position and an approximated position for grasping tissue between the tissue contacting surfaces;
an electromagnetic induction coil fixedly disposed within the first jaw member;
a thermal cutting element disposed at least partially within the electromagnetic induction coil and configured to protrude from the first jaw member and through the tissue contacting surface thereof, the thermal cutting element formed at least partially from an electromagnetic material capable of being inductively heated, and wherein the electromagnetic induction coil is adapted to connect to a source of energy to produce an electromagnetic field within the electromagnetic induction coil to thereby inductively heat the thermal cutting element; and
a cooling system disposed within the first jaw member proximate the thermal cutting element, the cooling system having a tube including:
a first conduit configured to convey compressed air from an external source therealong to absorb a first amount of heat from the thermal cutting element during or after activation thereof;
a second conduit configured to return the compressed air to a collection tank; and
an expansion nozzle disposed between the first conduit and the second conduit, the expansion nozzle configured to reduce the air pressure and increase the air velocity of the compressed air flowing from the first conduit to the second conduit allowing the second conduit to absorb a second amount of heat from the thermal cutting element during or after activation thereof.

11. The end effector assembly according to claim 10, wherein the tissue contacting surfaces are formed from an electrically-conductive material and adapted to connect to a source of energy for electrosurgically treating tissue grasped between the tissue contacting surfaces.

12. The end effector assembly according to claim 10, wherein the second conduit includes at least one orifice disposed therealong configured to remove smoke from the surgical site under suction.

13. The end effector assembly according to claim 12, wherein the at least one orifice includes a venturi-shaped opening for suctioning air from the surgical site.

14. The end effector assembly according to claim 10, wherein the thermal cutting element is formed at least partially from a ferromagnetic material.
